(19) United States
(12) Patent Application Publication
(10) Pub. No.: US 2004/0175701 A1
(43) Pub. Date: Sep. 9, 2004

(54) Treatment of silicon carbide to enhance binding ability

(76) Inventor: Yousef Haj-Ahmad, St. Catherines (CA)

Correspondence Address: Mark B. Eisen, c/o Dimock Stratton Clarizio LLP, Suite 3202, 20 Queen Street West, Box 102, Toronto, ON, M5H 3R3, CA

(21) Appl. No.: 10/379,383

(22) Filed: Mar. 5, 2003

(51) Int. Cl. C12Q 1/68; C01B 31/36

(52) U.S. Cl. 435/6; 423/345

(57) ABSTRACT

The present invention discloses methods for the processing of commercially obtained silicon carbide particles to enhance the nucleic acid binding properties of the silicon carbide. The methods use a variety of chemical washes which may also use high temperature treatment under gentle boiling followed by cooling and allowing the particles to settle, and milling to obtain the desired grit size.

### FIELD OF THE INVENTION

The invention relates generally to the field of processing of silicon carbide to enhance its nucleic acid binding properties.

### BACKGROUND OF THE INVENTION

It is known in that art that DNA will bind to silicon-containing materials such as glass slurries and diatomaceous earth. In fact, DNA purification kits are available which make use of these silicon-containing substances. For example, BioRad™ offers a plasmid purification kit using diatomaceous earth (Celite™) suspended in guanidine hydrochloride buffer.

U.S. Pat. Nos. 5,438,129 and 5,625,054 describe DNA isolation procedures which utilize fluorinated Celite™, fluorinated silicon dioxide or fluorinated aluminum hydroxide. These inventions require the use of toxic chemicals to create the fluorinated surfaces to which the DNA will bind. In addition, chaotropes which are also toxic are still required for these procedures.

U.S. Pat. No. 5,534,054 discloses the use of silicon tetrahydrazide for the purification of DNA, but the preparation of the binding material requires the use of toxic chemicals which can lead to conditions such as nausea and temporary blindness.

U.S. Pat. Nos. 6,177,278 and 6,291,248 disclose the use of silicon carbide, a dark grey or green, crystalline substance which is insoluble in water, acids and alkalis, to bind nucleic acids. Those patents disclose the use of a 15% weight/volume (w/v) slurry of silicon carbide prepared in either distilled water or a binding buffer of guanidine hydrochloride.

Several chemical factories around the world produce silicon carbide by combining silica with a carbon source, termed “coke”, at high temperatures reaching 2500° C. The resulting compound is ground into powders of various sizes in a particle size distribution denoted as “grit size”. The silicon carbide obtained from such manufacturers is usually fairly crude for biological applications

### SUMMARY OF THE INVENTION

It is now an object of the present invention to provide simple, inexpensive processing of silicon carbide to enhance its nucleic acid binding properties.

Accordingly the invention provides an inexpensive processing, and simple method of enhancing the binding properties of silicon carbide. The invention further provides for the use of silicon carbide having enhanced binding properties.

### DETAILED DESCRIPTION OF THE INVENTION

The present invention discloses the processing of commercially obtained silicon carbide particles to: (i) enhance the binding and elution of nucleic acids to silicon carbide; (ii) provide more uniform particle sizes; and (iii) to remove unwanted impurities. The processing of commercially obtained silicon carbide particles can be achieved using a variety of chemical washes which may use high temperature treatment under gentle boiling, followed by cooling and allowing the particles to settle. Supernatants from the settled particles represent waste materials that must be disposed of properly, including neutralization of an acid wash step. Some of the typical contaminants in commercially manufactured silicon carbide include silicon, silicon dioxide, carbon, iron, aluminium oxide, calcium oxide and sodium oxide.

Processing of silicon carbide to improve nucleic acid (DNA and RNA) binding and eluting properties of the silicon carbide can be achieved by washing the silicon carbide with a variety of solutions or through a series of washes. Typical wash solutions could include Tris (10 mM)/EDTA (1 mM) (TE buffer) from pH 7 to 8, water, hydrochloric acid, nitric acid, sodium hydroxide and ethanol.

Silicon carbide may function as a weakly acidic ion exchanger. The description ‘weakly’ refers to the Lewis concept of acid and base in that an acid is considered strong if it donates its proton more readily (like HCl) or weakly if the proton tends to hang around more (like acetic acid).

The processing of the silicon carbide can be assessed by measuring the quantity and quality of the nucleic acid which binds to the silicon carbide resin following processing, e.g. binding and elution of DNA. The quality of DNA was assessed using spectrophotometric analysis versus diphenylamine analysis of DNA quantity. The spectrophotometric analysis at 260 nm should reflect the DNA concentration based on the extinction coefficient of cesium chloride banded DNA, where one optical density (OD) is equal to 50 μg/mL. The diphenylamine analysis estimates the absolute DNA concentration based on a calorimetric reaction. The blue color development is measured in the visible region at 595/700 nm. When these two assessments are in agreement, the DNA quality is of the highest grade. DNA that is purified using a silicon carbide resin demonstrates an over-estimation of DNA concentration using spectrophotometric analysis.

DNA isolation using a silicon carbide packed column, was achieved using the DNA ethanol wash solution and DNA TE wash solution (above), which are passed through the resin in order to wash and elute DNA. Acid base treatment was also explored as an option for removal of trace impurities. Some solutions (e.g. 0.1% sodium dodecyl sulphate (SDS), 0.1 M sodium phosphate, 0.1×saline sodium citrate) do not assist in the processing of the silicon carbide and can reduce the efficacy of DNA isolation using silicon carbide.

Solutions for washing silicon carbide are optimal when they result in a high DNA yield and a low specphotometric to diphenylamine ratio. Some solutions such as EDTA and Tris-HCl result in low DNA yields, caused by a loss of DNA in the flow through and washes as well as extremely high spectrophotometer to diphenylamine ratios during column DNA purification. Other solutions, such as water and 95% ethanol, may produce satisfactory nucleic quality and quantity but can result in difficulty in sedimenting the silicon carbide during processing. The DNA yields using silicon carbide which had been pre-processed by washing with various solutions and measured using the diphenylamine assay are shown in graph 1.

[image]

The DNA spectrophotometric to diphenylamine ratios using silicon carbide which had been pre-processed by washing with various solutions (including 50% ethanol and 70% ethanol) are shown in graph 2. Graph 2 shows the data from three successive elutions (E1, E2 and E3).

[image]

The ideal numbers of washes will also be determined by the quality and quantity of the DNA isolated from silicon carbide pre-treated with various solutions. In addition, the clarity of the supernatant after each wash treatment can be an indicator of the ideal number of washes, with washing in a particular solution being halted once a clear supernatant was obtained. Combination washes of silicon carbide can also improve the quality and quantity of DNA isolated from pre-washed silicon carbide. Further, application of heat during the wash treatment can speed the processing of the silicon carbide. The heat treatment as compared to no heat application increased DNA yield noticeably and the spectrophotometric to diphenylamine ratio was lowered.

In one embodiment of the invention the processing of the silicon carbide particles comprises an ethanol water treatment, followed by three treatments with base, two inorganic acid treatments, washing with TE and water, followed by drying and roller milling.

For the ethanol-water treatment, 1.2-1.5 Kg of Showa Denko silicon carbide is slurried in 1.5 L of milliQ water in a 5 L beaker. The slurry is transferred to a four-necked 1.2 L round bottom flask using an addition funnel. The 5 L beaker and funnel are rinsed with an additional 0.5 L of milliQ water and the water added to the round bottom flask. 2 L of 95% ethanol are added to the flask. The slurry is heated with stirring to gentle boiling. Gentle boiling is maintained for about 30 minutes. While maintaining stirring, the heating is stopped, and the slurry allowed to cool to 75° C. The slurry can be cooled using an ice/water mix in a bowl in which the flask in inserted. When the slurry is cooled to 50° C., the stirring is stopped and the resin allowed to settle for about 30 minutes. After about 30 minutes, a discrete silicon carbide bed is formed, although the liquid above may be very grey and cloudy. The supernatant is waste and typically represents approximately 3-5% by mass of the initial silicon carbide. The supernatant is carefully aspirated and disposed of in an organic waste container. 4 L of distilled water are added to the silicon carbide bed and stirred for about 15 minutes and then the resin is allowed to settle for about 15 minutes. After about 15 minutes the supernatant is aspirated.

For the initial base treatment, 2 L of 0.5M sodium hydroxide (NaoH) is added to the flask containing the silicon carbide bed and then stirred to resuspend the silicon carbide cake. The base solution is heated to gentle boiling for about 40 minutes to about 85° C. and gentle boiling maintained for about 1 hour. The heat is then turned off, while maintaining stirring, and the slurry allowed to cool to 75° C. The slurry is then cooled to 50° C. using an ice/water mix in a bowl in which the flask in inserted. Once cooled, the stirring is stopped and the silicon carbide allowed to settle for 30 about minutes. Once settled, the silicon carbide bed should be well defined but may not be well consolidated. The supernatant is aspirated to a 4 L container to be neutralized and discarded. The silicon carbide cake is then washed by resuspension in 4 L of distilled water for about 15 minutes and then the resin is allowed to resettle for about 15 minutes and the supernatant aspirated. This washing is repeated twice more.

For the second base treatment 2 L of 0.5M NoaH is added to the silicon carbide and the resulting slurry stirred vigorously for about 30 minutes. No heating is applied in this step. After about 30 minutes, the stirring is stopped, the resin allowed to settle for about 15 minutes and the supernatant aspirated and discarded. The silicon carbide cake is then washed by resuspension in 4 L of distilled water for about 15 minutes and then the resin is allowed to resettle for about 15 minutes and the supernatant aspirated. This washing is repeated twice more.

The second base treatment and washing is then repeated.

The silicon carbide is then treated with nitric acid. 2 L of MilliQ water is added to the silicon carbide and stirred vigorously to resuspend the silicon carbide cake. The stirring is reduced, then about 363 ml of concentrated nitric acid is added to the silicon carbide slurry. The stirring is again increased and the acid solution heated for about 40 minutes to gentle boiling at about 85° C. Gentle boiling is then maintained at about 100° C. for about 1 hour. The heating is stopped, while stirring is continued, and the slurry allowed to cool to 85° C. The slurry is then cooled to 40° C. using an ice/water mix in a bowl in which the flask in inserted. After cooling to 40° C. the stirring is stopped and the silicon carbide particles allowed to settle for about 15 minutes. The acidic supernatant is aspirated and neutralized prior to disposal. 2 L of 0.5M NaoH is added to the silicon carbide cake and stirred for about 10 minutes. After about 10 minutes, the stirring is stopped and the resin allowed to settle for about 10 minutes before the supernatant is aspirated. The silicon carbide cake is then washed by resuspension in 4 L of distilled water for about 10 minutes and then the resin is allowed to resettle for about 10 minutes and the supernatant aspirated. This washing is repeated twice more.

For a second nitric acid wash, 2 L of distilled water is added to the silicon carbide and stirred vigorously to resuspend the silicon carbide. Then about 80 mL of concentrated nitric acid is carefully added to the slurry while continuously stirring for about 30 minutes. After about 30 minutes the stirring is stopped and the resin allowed to settle for about 15 minutes before aspirating the acidic supernatant for neutralization prior to disposal. 2 L of 0.5M NaoH is added to the silicon carbide cake and stirred for about 10 minutes. After about 10 minutes, the stirring is stopped and the resin allowed to settle for about 10 minutes before the supernatant is aspirated. The silicon carbide cake is then washed by resuspension in 4 L of distilled water for about 10 minutes and then the resin is allowed to resettle for about 10 minutes and the supernatant aspirated. The silicon carbide cake is then washed again by resuspension in 2 L of distilled water for about 10 minutes and then the resin is allowed to resettle for about 10 minutes and the supernatant aspirated. Following acid treatment, the silicon carbide forms a cake much faster than prior to acid treatment.

The silicon carbide particles are then washed by resuspension in about 2 L of TE buffer, pH 11 and stirred for about 15 minutes. After about 15 minutes the stirring is stopped and the resin allowed to settle for about 10 minutes, following which the supernatant is discarded. 4 L of MilliQ water is added to the silicon carbide resin and stirred for about 15 minutes to resuspend the resin. After about 15 minutes the stirring is stopped and the silicon carbide resin allowed to settle for about 10 minutes before the supernatant is aspirated. 2 L of MilliQ water is added to the silicon carbide resin and stirred for about 15 minutes to resuspend the resin. After about 15 minutes the stirring is stopped and the silicon carbide resin allowed to settle for about 10 minutes before the supernatant is aspirated.

The washing in TE is followed by rinsing in MilliQ water and is repeated twice more.

1 L of MilliQ water is added to the silicon carbide resin cake, and stirred for about 15 minutes to resuspend the resin. The resuspension is then aspirated into 2 Pyrex pans and allowed to settle for about 15 minutes. Once the resin is settled, the supernatant is discarded and the Pyrex trays placed in a 65° C. oven and allowed to dry for about 24 hours. On completion of the drying the silicon carbide powder is cooled and transferred to polypropylene rolling bottles for milling for about 6 to 12 hours. The duration of the milling determines the particle size distribution. Increasing the uniformity of the resin particles directly enhances flow rate in a packed column. The washed resin decreases the amount of loose particulate material making the particle size distribution narrower (smaller standard deviation) and uniform as shown in Graphs 3 and 4.

[image]

Various embodiments of the present invention having been thus described in detail by way of example, it will be apparent to those skilled in the art that variations and modifications may be made without departing from the invention. The invention includes all such variations and modifications as fall within the scope of the appended claims.

What is claimed is:

1. A method of processing manufactured silicon carbide to enhance its nucleic acid binding properties comprising washing the silicon carbide with a mixture of alcohol and water.

2. The method of claim 1 wherein the alcohol is a primary alcohol.

3. The method of claim 1 wherein the alcohol is selected from methanol, ethanol, and propanol.

4. The method of claim 1 wherein the alcohol is ethanol.

5. The method of claim 1 wherein the mixture is from 70% to 95% ethanol.

6. The method of claim 1 wherein the silicon carbide is heated during washing.

7. A method of processing manufactured silicon carbide to enhance its nucleic acid binding properties comprising washing the silicon carbide with an inorganic acid.

8. A method of processing manufactured silicon carbide to enhance its nucleic acid binding properties comprising washing the silicon carbide with an inorganic acid selected from hydrochloric acid, nitric acid and a mixture of hydrochloric and nitric acids.

9. A method of processing manufactured silicon carbide to enhance its nucleic acid binding properties comprising washing the silicon carbide at least once with an inorganic base, and then washing the silicon carbide at least once with an inorganic acid.

10. The method of claim 9 wherein the inorganic base is sodium hydroxide and the inorganic acid is selected from hydrochloric acid, nitric acid and a mixture of hydrochloric and nitric acids.

11. A method of processing manufactured silicon carbide to enhance its nucleic acid binding properties comprising washing the silicon carbide at least once with a mixture of alcohol and water, washing the silicon carbide at least once with an inorganic base, and then washing the silicon carbide at least once with an inorganic acid.

12. The method of claim 11 wherein the mixture of alcohol and water is ethanol in a concentration of from 50% (v/v) to 95% (v/v).

13. The method of claim 11 wherein the inorganic base is sodium hydroxide.

14. The method of claim 11 wherein the inorganic acid is selected from hydrochloric acid, nitric acid and a mixture of hydrochloric and nitric acids.

15. The method of claim 11 wherein the mixture of alcohol and water is ethanol in a concentration of from 50% (v/v) to 95% (v/v), the inorganic base is sodium hydroxide, and the inorganic acid is selected from hydrochloric acid, nitric acid and a mixture of hydrochloric and nitric acids.

16. The method of claim 11 wherein the silicon carbide is heated during at least one washing.

17. The method of claim 11 wherein the silicon carbide is subsequently milled to the desired grit size.

18. The use of silicon carbide treated according to the method of claim 17 for binding nucleic acid.
